(19) United States
(12) Patent Application Publication
(10) Pub. No.: US 2014/0081084 A1
(43) Pub. Date: Mar. 20, 2014

(54) SHARED BITE BLOCK

(71) Applicants: Dawn A. Domenico, Goffstown, NH (US); Anthony J. Domenico, Menifee, CA (US); James F. Dana, II, Bedford, NH (US)

(72) Inventors: Dawn A. Domenico, Goffstown, NH (US); Anthony J. Domenico, Menifee, CA (US); James F. Dana, II, Bedford, NH (US)

(21) Appl. No.: 13/943,928

(22) Filed: Jul. 17, 2013

Related U.S. Application Data
(60) Provisional application No. 61/702,638, filed on Sep. 18, 2012.

(51) Int. Cl. A61B 1/00; A61B 1/273; A61M 16/04

(52) U.S. Cl. CPC A61B 1/00154; A61M 16/0497; A61B 1/2733
USPC 600/114; 128/200.26; 29/428

(57) ABSTRACT

A shared bite block is disclosed which can be employed to simultaneously establish a viable airway for a patient, while providing surgical access to the esophagus of the patient. Structurally, the device includes a hollow, tubular shaped body portion that is formed as a sidewall with open ends. The sidewall surrounds an interior space and a partition extends across the interior space to divide the space and establish a hole and a guideway. A pair of extensions is each attached to the proximal end of the body portion and a flange is formed at the distal end of the body portion. In use, the patient bites down on the body portion between the extensions and the flange. An airway tube can then be inserted into the guideway of the body portion. Unobstructed surgical access into the esophagus is established through the hole of the body portion of the device.

This application claims priority to U.S. Provisional Application No. 61/702,638, filed Sep. 18, 2012, to Dawn Domenico and Anthony J. Domenico, titled “SHARED BITE BLOCK,” the entire contents of which is hereby incorporated by reference herein.

### FIELD OF THE INVENTION

The present invention pertains generally to medical devices. More particularly, the present invention pertains to medical devices which provide surgical access to the esophagus of a patient. The present invention is particularly, but not exclusively, useful as bite block that allows for an insertion of an airway tube into a patient's pharynx and provides surgical access to the esophagus of a patient.

### BACKGROUND OF THE INVENTION

There are several common medical procedures in which a surgical instrument or probe is introduced into a patient's mouth, advanced through the patient's pharynx and inserted into the patient's esophagus. For these procedures, a mouth guard is generally employed to protect the patient's teeth and the surgical instrument. In order to perform these types of procedures patients are given medication to blunt the gag reflex. These medications range from topical local anesthesia to those that cause deep sedation and possibly general anesthesia.

One such procedure which requires access to the esophagus is the so-called esophagogastroduodenoscopy procedure. In this procedure, an endoscope is introduced, as described above, and used to examine the lining of esophagus, the stomach and/or the duodenum. In some cases, biopsies can be taken. If a narrowing of the esophagus is present, a surgical instrument can also be used to stretch or widen the affected area.

Another common procedure which requires access to the esophagus is the so-called transesophageal echocardiogram procedure. This procedure takes advantage of the fact that the esophagus passes in close proximity to the heart. In this procedure, an ultrasonic probe is introduced into the esophagus, as described above, and a sonogram of the heart is produced.

In all these procedures, it is important to establish and maintain the patency of a patient's airway. For this purpose, the distal end of an airway tube can be inserted into the patient's upper respiratory tract and then carefully advanced into the oropharyngeal space. Access to the upper respiratory tract is typically achieved through the patient's mouth.

In light of the above it is an object of the present invention to provide a bite block which protects a patient's teeth during a procedure in which a surgical instrument is introduced into the patient's esophagus. Another object of the present invention is to provide a bite block which accommodates an airway tube during a procedure in which a surgical instrument is introduced into the patient's esophagus. Still another object of the present invention is to provide a shared bite block and corresponding methods of use that are easy to use and comparatively cost effective.

### SUMMARY OF THE INVENTION

In accordance with the present invention, a device (i.e. a “Shared Bite Block”) is disclosed which can be employed to establish a viable airway for a patient, while simultaneously providing surgical access to the esophagus of the patient. In its overall effect, the device of the present invention is a platform which guides the insertion of an airway tube into the pharynx for placement of the tube in breathing communication with the patient's trachea. At the same time, it also allows for the access of surgical implements (e.g. an endoscope or transesophageal echocardiography probe) through the pharynx and into the esophagus. In particular, the present invention is intended for use in an esophagogastroduodenoscopy procedure and transesophageal echocardiogram.

Structurally, the device of the present invention includes a hollow, tubular shaped body portion which is essentially formed as a sidewall with open ends. The body portion defines an axis and it has a proximal end and a distal end. Further, the sidewall is centered on the axis, and it is oriented substantially parallel to the axis to surround an interior space.

A partition extends across the interior space of the body portion, and extends between the proximal end and the distal end of the body portion, to divide the interior space into two parts. Specifically, this division establishes a hole through the interior space on one side of the partition, and it establishes a guideway through the interior space on the other side of the partition.

The device of the present invention also includes a pair of extensions which are each attached to the proximal end of the body portion. Further, these extensions protrude outwardly from the proximal end of the body portion, and away from the axis in diametrically opposite directions. A flange is formed at the distal end of the body portion. Additionally, a strap is provided to hold the device on the head of a patient during a surgical procedure. In detail, one end of the strap is attached to one of the extensions, and the other end of the strap is attached to the other extension. Functionally, the strap acts to hold the extensions against the cheeks of the patient, with the lips and teeth of the patient between the extensions and the flange, to secure the device on the head of the patient.

With the body portion (i.e. Shared Bite Block) in place on a patient, an airway tube can then be selectively inserted into the guideway of the body portion. Preferably, this can be done to position the proximal end of the airway tube substantially flush with the proximal end of the body portion and, thus, not obstruct access into the hole of the body portion. Once the airway tube is in place, the distal end of the airway tube will be positioned in breathing communication with the trachea above the glottic opening of the patient. Importantly, unobstructed surgical access into the esophagus is established through the hole of the body portion of the device.

In greater detail, the airway tube is preferably formed with a tab at its proximal end for use in positioning the airway tube in the guideway. Also, the airway tube may be configured to bias the distal end of the airway tube in a predetermined direction toward the trachea, after the airway tube is inserted into the guideway. Further, in a preferred embodiment of the present invention, the guideway is inclined toward the axis at a tilt angle φ to help center the airway tube for its placement at the glottic opening of the trachea. Specifically, this tilt is done with the guideway being closest to the axis at the distal end of the body portion. In general, the tilt angle φ is in a range between 5° and 15°. As envisioned for the present invention, the airway tube is made of a flexible medical grade polymer. Also, in cooperation with the airway tube, the hole in the body portion of the device is dimensioned to receive an endoscope. More specifically, the hole is oblong shaped with a short dimension of about 0.83 inch and a long dimension of about 1.0 inch.

In an alternate embodiment of the present invention, a sensor access port is created by a divider that extends in the interior space between the partition and the sidewall, and between the proximal end and the distal end of the body portion. Typically, the sensor access port will have a circular cross-section and will be dimensioned to selectively receive a variety of different sensor probes. In any event, the body portion will preferably be made of a medical grade plastic.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a perspective view of a device (i.e. a Shared Bite Block) in accordance with the present invention;

FIG. 2 is a cross-section view of the device as seen along the line 2-2 in FIG. 1;

FIG. 3 is a side view of an airway tube as used with the present invention;

FIG. 4 illustrates the positioning of a device of the present invention in the mouth of a patient with an airway tube inserted through the guideway of the device to establish breathing communication with the patient's trachea; and

FIG. 5 is a view of the device as shown in FIG. 2 with an airway tube inserted into the guideway of the device and an endoscope positioned through the hole of the device to perform a surgical procedure.

### DESCRIPTION OF THE PREFERRED EMBODIMENTS

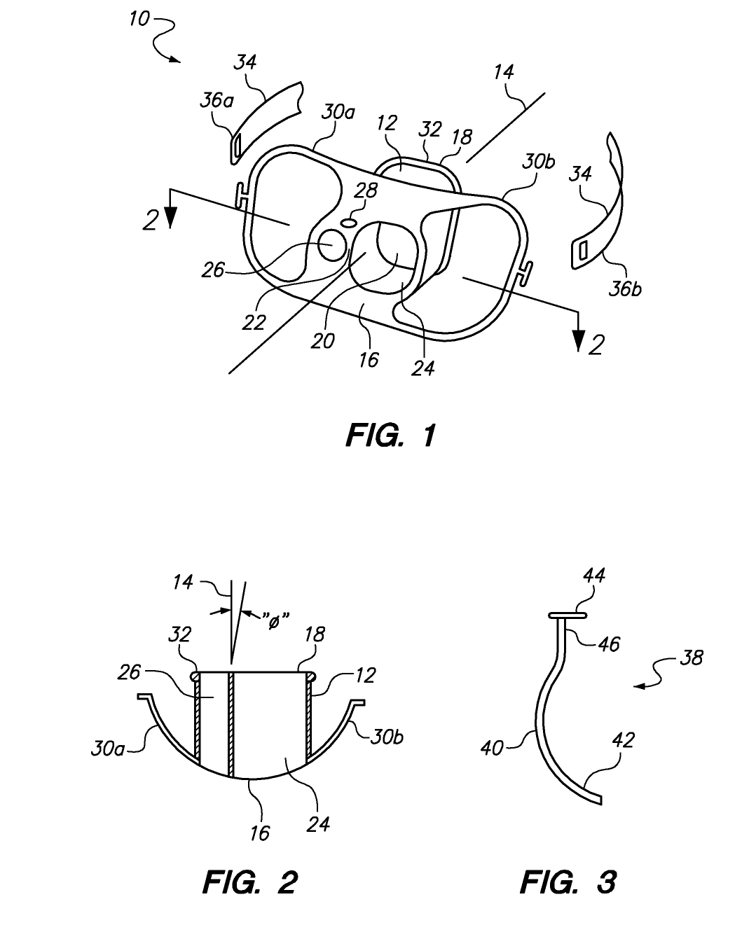

With initial reference to FIG. 1, a device (i.e. a “Shared Bite Block”) is shown and generally designated 10. As shown, the device 10 includes a hollow, tubular shaped body portion 12 which is essentially formed as a sidewall. It can further be seen that the body portion 12 defines an axis 14 and it has an open proximal end 16 and an open distal end 18. With this cooperative arrangement, the sidewall of the body portion 12 is centered on the axis 14, and it is oriented substantially parallel to the axis 14 to surround an interior space 20.

Continuing with FIG. 1, it can be seen that a partition 22 extends across the interior space 20 of the body portion 12, and extends between the proximal end 18 and the distal end 16 of the body portion 12. Functionally, the partition 22 divides the interior space 20. Specifically, this division establishes a hole 24 through the interior space 20 on one side of the partition 22, and it establishes a guideway 26 through the interior space 20 on the other side of the partition 22. FIG. 1 also shows that a sensor access port 28 can also be included in the interior space 20.

As best appreciated by cross-referencing FIG. 1 with FIG. 2, it can be seen that the device 10 includes a pair of extensions 30a,b which are each attached to the proximal end 16 of the body portion 12. As further shown, the extensions 30a,b protrude outwardly from the proximal end 16 of the body portion 12, and away from the axis 14 in diametrically opposite directions. FIGS. 1 and 2 both show that a flange 32 is formed at the distal end 18 of the body portion 12. Additionally, as best seen in FIG. 1, a strap 34 can be provided to hold the device 10 on the head of a patient during a surgical procedure. In detail, end 36a of the strap 34 is attached to extension 30a and the end 36b of the strap 34 is attached to extension 30b. Functionally, the strap 34 acts to hold the extensions against the cheeks of the patient, with the lips and teeth of the patient between the extensions 30a,b and the flange 32, to secure the device 10 on the head of the patient.

FIG. 3 shows an example of an airway tube (generally designated 38) for use in the present invention. As shown in side-view, the airway tube 38 includes a tube 40 that is bent or curved into a suitable configuration to allow the practitioner to advance the distal end 42 of the tube through the larynx and into the trachea. As shown, a tab 44 can be attached to the proximal end 46 of the tube to facilitate the positioning of the tube 40.

Cross-referencing FIGS. 2 and 3, it can be seen that the distal end 42 of the airway tube 38 is sized to pass through the guideway 26. Moreover, as shown, the guideway 26 can be inclined toward the axis 14 (shown in FIG. 1) at a tilt angle φ to help center the airway tube 38 for its placement at the glottic opening of the patient's trachea. Specifically, this tilt is done with the guideway 26 being closest to the axis 14 at the distal end 18 of the body portion 12. In general, the tilt angle φ is in a range between 5° and 15°. As envisioned for the present invention, the airway tube 38 is made of a flexible medical grade polymer.

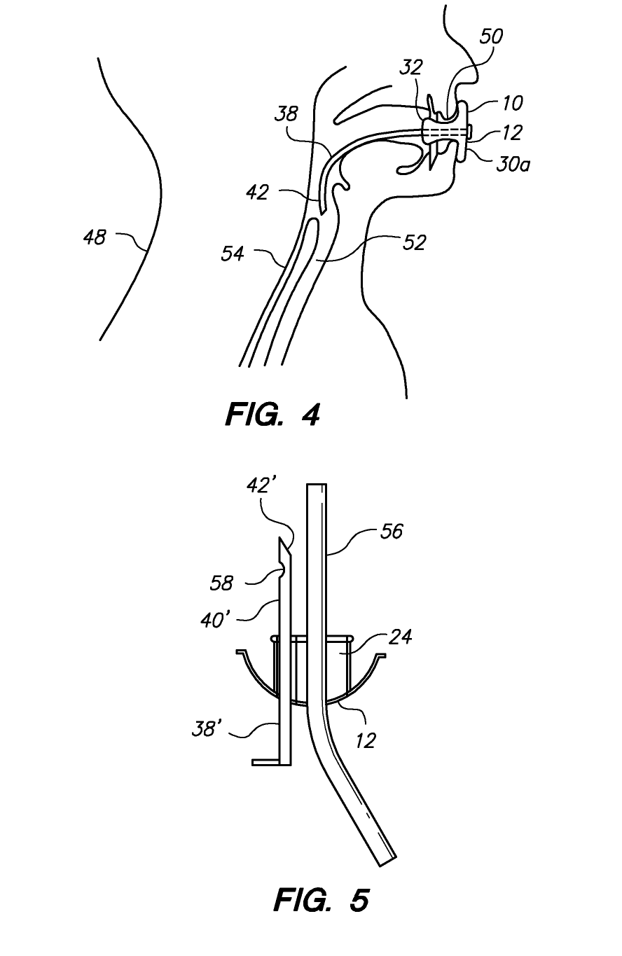

FIG. 4 shows the device 10 operationally positioned in a patient 48. During a procedure, the body portion 12 is positioned in the patient's mouth with the patient's lips 50 positioned between the flange 32 and extensions 30a,b as shown in FIG. 4. With the body portion 12 in place on the patient 48, an airway tube 38 can then be selectively inserted into the guideway 26 (see FIG. 1 or 2) of the body portion 12. Preferably, this is done to position the proximal end 46 of the airway tube 38 (FIG. 3) substantially flush with the proximal end 18 of the body portion 12 (FIG. 2) and, thus, not obstruct access into the hole 24 of the body portion 12. FIG. 4 shows the airway tube 38 in place with the distal end 42 of the airway tube 38 will be positioned in breathing communication with the trachea 52 above the glottic opening of the patient 48. With this arrangement, FIG. 4 shows that unobstructed surgical access into the esophagus 54 is established through the hole 24 (FIG. 2) of the body portion 12.

FIG. 5 illustrates that an endoscope 56 can be passed through the hole 24 during a procedure. For example, an endoscope 56 or transesophageal echocardiography probe can be advanced through the pharynx and into the esophagus 54 (FIG. 4). In particular, the present invention is intended for use in an esophagogastroduodenoscopy procedure and transesophageal echocardiogram. FIG. 5 also shows another embodiment of an airway tube 38′ having a tube 40′ with a tapered distal end 42′ to facilitate transit through the larynx. From FIG. 5, it can be seen that an opening 58 can be formed opposite the tapered end 42′ to maintain fluid communication in the event the tapered end 42′ becomes blocked.

While the particular Shared Bite Block as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A device for establishing surgical access into the esophagus of a patient which comprises:
a hollow tubular shaped body portion defining an axis and having a proximal end and a distal end, with a sidewall oriented substantially parallel to the axis and extending between the proximal end and the distal end of the body portion to surround an interior space, with the sidewall substantially centered on the axis;
a partition extending across the interior space between the proximal end and the distal end of the body portion to establish a hole through the interior space on a first side of the partition, and to establish a guideway through the interior space on a second side of the partition; and
an airway tube having a proximal end and a distal end, wherein the airway tube is selectively inserted into the guideway of the body portion, to position the proximal end of the airway tube substantially flush with the proximal end of the body portion, and to position the distal end of the airway tube in breathing communication with the trachea of the patient, while establishing surgical access into the esophagus through the hole of the body portion of the device.

2. A device as recited in claim 1 wherein the device is a bite block and further comprises:
a first extension and a second extension, with each extension attached to the proximal end of the body portion, and with each extension protruding outwardly from the body portion and away from the axis in a diametrically opposite direction from the other extension;
a flange formed at the distal end of the body portion; and
a strap having a first end attached to the first extension and a second end attached to the second extension to hold the bite block on the head of the patient with the first and second extensions against the cheeks of the patient and with the lips and teeth of the patient between the extensions and the flange.

3. A device as recited in claim 1 wherein the airway tube is formed with a tab at the proximal end thereof for use in positioning the airway tube in the guideway, and wherein the distal end of the airway tube is formed with a taper and has an opening formed opposite the taper to avoid blockage of the airway tube.

4. A device as recited in claim 1 wherein the airway tube is configured to bias the distal end of the airway tube in a predetermined direction toward the trachea, after the airway tube is inserted into the guideway.

5. A device as recited in claim 4 wherein the airway tube is made of a flexible medical grade polymer.

6. A device as recited in claim 1 wherein the guideway is inclined toward the axis at a tilt angle (I), with the guideway being closest to the axis at the distal end of the body portion.

7. A device as recited in claim 6 wherein the tilt angle φ is in a range between 5° and 15°.

8. A device as recited in claim 1 further comprising a divider extending in the interior space between the partition and the sidewall, and between the proximal end and the distal end of the body portion to create a sensor access port.

9. A device as recited in claim 1 wherein the hole is dimensioned to receive an endoscope therethrough.

10. A device as recited in claim 9 wherein the hole is oblong shaped with a short dimension of about 0.83 inch and a long dimension of about 1.0 inch.

11. A device as recited in claim 1 wherein the body portion is made of a medical grade plastic.

12. A device for establishing surgical access into the esophagus of a patient which comprises:
a hollow, tubular shaped body portion defining an axis and having a proximal end and a distal end, with a sidewall oriented substantially parallel to the axis and extending between the proximal end and the distal end of the body portion to surround an interior space, with the sidewall substantially centered on the axis;
a partition extending across the interior space between the proximal end and the distal end of the body portion to establish a hole through the interior space on a first side of the partition, and to establish a guideway through the interior space on a second side of the partition; and
a divider extending in the interior space between the partition and the sidewall, and between the proximal end and the distal end of the body portion to create a sensor access port, and further wherein the body portion is made of a medical grade plastic.

13. A device as recited in claim 12 further comprising an airway tube having a proximal end and a distal end, wherein the airway tube is selectively inserted into the guideway of the body portion, to position the proximal end of the airway tube substantially flush with the proximal end of the body portion, and to position the distal end of the airway tube in breathing communication with the trachea of the patient, while establishing surgical access into the esophagus through the hole of the body portion of the device.

14. A device as recited in claim 13 wherein the device is a bite block and further comprises:
a first extension and a second extension, with each extension attached to the proximal end of the body portion, and with each extension protruding outwardly from the body portion and away from the axis in a diametrically opposite direction from the other extension;
a flange formed at the distal end of the body portion; and
a strap having a first end attached to the first extension and a second end attached to the second extension to hold the bite block on the head of the patient with the first and second extensions against the cheeks of the patient and with the lips and teeth of the patient between the extensions and the flange.

15. A device as recited in claim 13 wherein the airway tube is formed with a tab at the proximal end thereof for use in positioning the airway tube in the guideway, wherein the airway tube is configured to bias the distal end of the airway tube in a predetermined direction toward the trachea, after the airway tube is inserted into the guideway, and wherein the airway tube is made of a flexible medical grade polymer.

16. A device as recited in claim 13 wherein the guideway is inclined toward the axis at a tilt angle φ, with the guideway being closest to the axis at the distal end of the body portion, and wherein the tilt angle φ is in a range between 5° and 15°.

17. A device as recited in claim 13 wherein the hole is dimensioned to receive an endoscope therethrough, and wherein the hole is oblong shaped with a short dimension of about 0.83 inch and a long dimension of about 1.0 inch.

18. A method for manufacturing a device for establishing surgical access into the esophagus of a patient which comprises the steps of:
providing a hollow, tubular shaped body portion made of a medical grade plastic, wherein the body portion defines an axis and has a proximal end and a distal end, with a sidewall oriented substantially parallel to the axis and extending between the proximal end and the distal end of the body portion to surround an interior space, with the sidewall substantially centered on the axis, wherein a partition extends across the interior space between the proximal end and the distal end of the body portion to establish a hole through the interior space on a first side of the partition and to establish a guideway through the interior space on a second side of the partition, wherein the guideway is inclined toward the axis at a tilt angle φ, and wherein a divider extends in the interior space between the partition and the sidewall, and between the proximal end and the distal end of the body portion to create a sensor access port, and further wherein the body portion is formed with a flange at the distal end;
affixing a first extension and a second extension, respectively, to the proximal end of the body portion with each extension protruding outwardly from the body portion and away from the axis in a diametrically opposite direction from the other extension; and
attaching a first end of a strap to the first extension and a second end of the strap to the second extension to hold the bite block on the head of the patient with the first and second extensions against the cheeks of the patient and with the lips and teeth of the patient between the extensions and the flange.

19. A method as recited in claim 18 further comprising the steps of:
dimensioning the guideway to receive an airway tube therethrough, wherein the airway tube has a proximal end and a distal end, wherein the airway tube is selectively inserted into the guideway of the body portion, to position the proximal end of the airway tube substantially flush with the proximal end of the body portion, and to position the distal end of the airway tube in breathing communication with the trachea of the patient, while establishing surgical access into the esophagus through the hole of the body portion of the device;
forming the airway tube with a tab at the proximal end thereof for use in positioning the airway tube in the guideway; and
configuring the airway tube to bias the distal end of the airway tube in a predetermined direction toward the trachea, after the airway tube is inserted into the guideway, and wherein the airway tube is made of a flexible medical grade polymer.

20. A method as recited in claim 18 wherein the hole is dimensioned to receive an endoscope therethrough, and wherein the hole is oblong shaped with a short dimension of about 0.83 inch and a long dimension of about 1.0 inch.
